(19) United States
(12) Patent Application Publication
(10) Pub. No.: US 2026/0341416 A1
(43) Pub. Date: Oct. 29, 2026

(54) NUCLEIC-ACID-MOLECULE-NETWORK PROCESSING DEVICE AND NUCLEIC-ACID-MOLECULE-NETWORK PROCESSING METHOD

(71) Applicant: Mitsubishi Electric Corporation, Tokyo (JP)

(72) Inventor: Hiroshi KAGE, Tokyo (JP)

(73) Assignee: Mitsubishi Electric Corporation, Tokyo (JP)

(21) Appl. No.: 19/109,135

(22) PCT Filed: Sep. 15, 2022
(86) PCT No.: PCT/JP2022/034530

§ 371 Date: Mar. 6, 2025

(51) Int. Cl. G16B 50/30; G16B 30/00; G16B 40/20

(52) U.S. Cl. CPC G16B 50/30; G16B 30/00; G16B 40/20

(57) ABSTRACT

A nucleic-acid-molecule-network processing device includes; a training-input-data specifying unit that specifies training input data; a training-output-data specifying unit that specifies training output data corresponding to the training input data; and a nucleic-acid-molecule associative-memory training unit that trains a learning network, which is a neural circuit network including nucleic acid molecules, to make an inference of the training output data from the training input data when a nucleic acid molecule solution expressing the training input data in concentration is input and to output a nucleic acid molecule solution expressing the training output data in concentration.

### TECHNICAL FIELD

The disclosure relates to a nucleic-acid-molecule-network processing device and a nucleic-acid-molecule-network processing method.

### BACKGROUND ART

The rapid increase in the amount of data distributed on the Internet has led to a serious problem of information explosion. To address this problem, data centers are being built in various parts of the world.

A large number of hard disk drives (HDDs) are used as storage devices in such data centers. However, today, the recording density of HDDs has reached its technological limit, so there is no other way to deal with the problem of information explosion than to increase the number of data centers.

In recent years, video distribution has been increasing exponentially; therefore, it is expected that no matter how many more data centers are built, it will not be possible to cover the information explosion. For this reason, deoxyribonucleic acid (DNA) is attracting attention as a storage device that can record data at higher density than HDDs. For example, PTL 1 discloses a technique for constructing data storage with DNA.

### PRIOR ART REFERENCE

### Patent Reference

Patent Literature 1: Japanese Patent Application (Translated PCT Application) No. 2021-518164

### SUMMARY OF THE INVENTION

### Problem to be Solved by the Invention

However, with Conventional technology, it is necessary to specify base sequences on DNA by using a DNA sequencer in order to read data from a DNA storage, which is a recording device using DNA. Since reading data per base takes a DNA sequencer several tens of minutes, it takes a very long time to read a large amount of data.

Accordingly, it is an object of one or more aspects of the disclosure to enable data to be read at a higher speed from a storage composed of nucleic acid molecules.

### Means of Solving the Problem

A nucleic-acid-molecule-network processing device according to a first aspect pf the disclosure includes: a training-input-data specifying unit to specify training input data; a training-output-data specifying unit to specify training output data corresponding to the training input data; and a training unit to train a learning network to infer the training output data from the training input data when a nucleic acid molecule solution expressing the training input data in concentration is input and to output a nucleic acid molecule solution expressing the training output data in concentration, the learning network being a neural circuit network including nucleic acid molecules.

A nucleic-acid-molecule-network processing device according to a second aspect of the disclosure includes: a processing-input-data specifying unit to specify processing input data; a network holding unit to input a nucleic acid molecule solution expressing the processing input data in concentration to a processing network, the processing network being a neural circuit network including nucleic acid molecules reflecting a result obtained through training of a learning network, the learning network being a neural circuit network including nucleic acid molecules, the learning network being trained to make an inference of training output data corresponding to training input data from the training input data when a nucleic acid molecule solution expressing the training input data in concentration is input, and to output a nucleic acid molecule solution expressing the training output data in concentration; and an output-data acquiring unit to acquire data expressed in concentration of a nucleic acid molecule solution output from the processing network as output data.

A nucleic-acid-molecule-network processing method according to a first aspect of the disclosure includes: specifying training input data; specifying training output data corresponding to the training input data; and training a learning network to make an inference of the training output data from the training input data when a nucleic acid molecule solution expressing the training input data in concentration is input and to output a nucleic acid molecule solution expressing the training output data in concentration, the learning network being a neural circuit network including nucleic acid molecules.

A nucleic-acid-molecule-network-processing method according to a second aspect of the disclosure includes: specifying processing input data; inputting a nucleic acid molecule solution expressing the processing input data in concentration to a processing network, the processing network being a neural circuit network including nucleic acid molecules reflecting a result obtained through training of a learning network, the learning network being a neural circuit network including nucleic acid molecules, the learning network being trained to make an inference of training output data corresponding to training input data from the training input data when a nucleic acid molecule solution expressing the training input data in concentration is input, and to output a nucleic acid molecule solution expressing the training output data in concentration; and acquiring data expressed in concentration of a nucleic acid molecule solution output from the processing network as output data.

### Effects of the Invention

According to one or more aspects of the present disclosure, data can be read at a higher speed from a storage composed of nucleic acid molecules.

### BRIEF DESCRIPTION OF THE DRAWINGS

FIG. 1 is a block diagram schematically illustrating a configuration of a nucleic-acid-molecule-data storage device according to a first embodiment.

FIG. 2 is a schematic diagram illustrating an example of a neural circuit network model.

FIG. 3 is a schematic diagram illustrating an implementation example of a CSTR.

FIG. 4 is a schematic diagram illustrating an example of a configuration in which a CSTR is modeled.

FIG. 5 is a block diagram schematically illustrating a configuration of a nucleic-acid-molecule-data storage device according to a second embodiment.

FIG. 6 is a schematic diagram illustrating a correspondence table for binarizing each alphabetic character into 5 bits.

FIGS. 7A and 7B are schematic diagrams illustrating time-series binarization patterns of training input data in supervised data composed of 26 English words, each consisting of 9 characters.

FIGS. 8A and 8B are schematic diagrams illustrating time-series binarization patterns of training output data of supervised data composed of 26 English words, each consisting of 9 characters.

FIGS. 9A to 9C are schematic diagrams for describing the processing input data and the output data in a third embodiment,

FIGS. 10A and 10B are schematic diagrams for explaining a sigmoid function.

FIG. 11 is a schematic diagram illustrating an example of a binary signal indicating “alcoholic.”

FIGS. 12A and 12B are schematic diagrams illustrating an example of training input data and training output data according to a fourth embodiment.

FIGS. 13A to 13C are schematic diagrams for describing the processing input data and the output data in a fourth embodiment,

FIG. 14 is a schematic diagram illustrating an example of an English word that has been restored by performing multiple runs of restoration.

### MODE FOR CARRYING OUT THE INVENTION

### First Embodiment

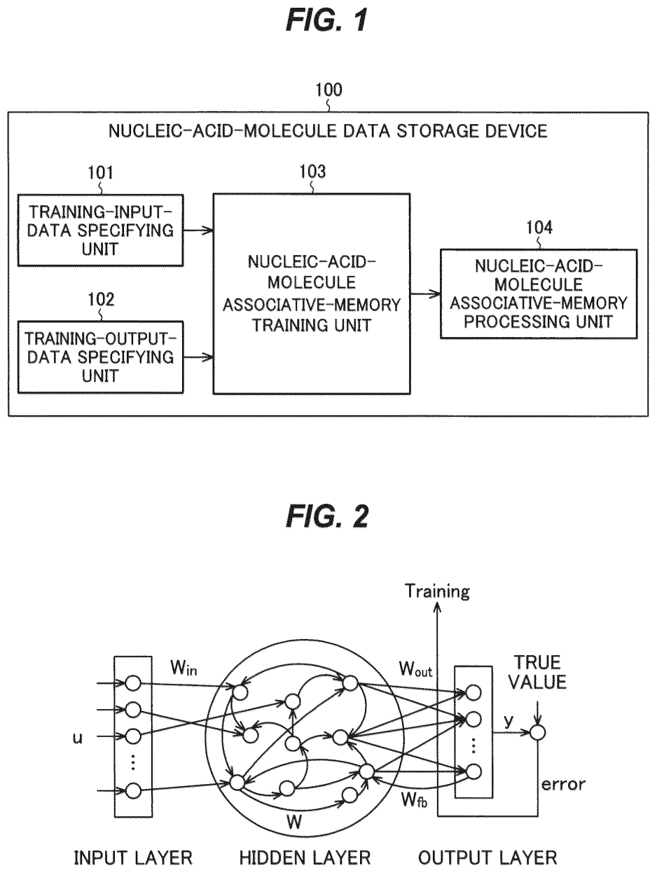

FIG. 1 is a block diagram schematically illustrating a configuration of a nucleic-acid-molecule-data storage device 100 serving as a nucleic-acid-molecule-network processing device according to the first embodiment.

The nucleic-acid-molecule-data storage device 100 includes a training-input-data specifying unit 101, a training-output-data specifying unit 102, a nucleic-acid-molecule associative-memory training unit 103, and a nucleic-acid-molecule associative-memory processing unit 104.

The training-input-data specifying unit 101 specifies training input data that is input data serving as training data. For example, the training-input-data specifying unit 101 generates a nucleic acid molecule solution that numerically expresses training input data in concentration and gives the nucleic acid molecule solution to a nucleic-acid-molecule associative-memory training unit 103.

The training-output-data specifying unit 102 specifies training output data that is output data serving as truth data. In other words, the training-output-data specifying unit 102 specifies training output data corresponding to the training input data and serving as truth data of the training input data. Specifically, as will be described later, the training-output-data specifying unit 102 gives an M-dimensional vector Y_{target} as the training output data to the nucleic-acid-molecule associative-memory training unit 103.

The nucleic-acid-molecule associative-memory training unit 103, which is a training unit that trains a learning network that is a neural circuit network composed of nucleic acid molecules, to estimate training output data from training input data in response to receiving a nucleic acid molecule solution expressing the training input data in concentration and to output a nucleic acid molecule solution expressing the training output data in concentration.

For example, the nucleic-acid-molecule associative-memory training unit 103 performs associative memory training on a learning network to constructs a neural circuit network that outputs training output data specified by the training-output-data specifying unit 102 in response to receiving training input data specified by the training-input-data specifying unit 101.

In the first embodiment, the nucleic-acid-molecule associative-memory training unit 103 follows a training algorithm of an echo state network (ESN), a type of reservoir computing based on discrete time, which is a variety of recurrent neural networks, to perform associative memory training on a neural circuit network composed of nucleic acid molecules, or DNA. The above operation will now be explained.

First, how calculations can be performed by using DNA is explained in three points.

As a first point, a neural circuit network model and the substance of signal processing will be described.

A neural circuit network model can be represented as a three-layer neural network illustrated in FIG. 2.

The neural circuit network model consists of an input layer, a hidden layer, and an output layer. The multiple units within the hidden layer are interconnected. The neural circuit network model according to the first embodiment has connections from the units in the output layer to the units in the hidden layers, which is different from network models frequently used in the realm of deep learning in which the input layer, the hidden layer, and the output layer only have feedforward connections.

On the basis of such a neural circuit network model, the nucleic-acid-molecule associative-memory training unit 103 proceeds with training in accordance with an ESN training algorithm and stores the obtained results as weights between the units.

Next, the substance of signal processing will be described.

Particularly, the signal processing for unit output in each layer is carried out by DNA chemical reactions, and the numerical values that constitute the substance of the signals are determined by the concentration of DNA. The multiplication of inter-unit weights for a signal can be implemented by multiplication operation using a DNA strand displacement reaction, as described in Reference 1 below.
Reference 1: Zou et al., “Four-Analog Computation Based on DNA Strand Displacement,” ACS Omega 2 (8), pp. 4143-4160, 2017.

Next, as a second point, how to construct a neural circuit network with DNA will be explained.

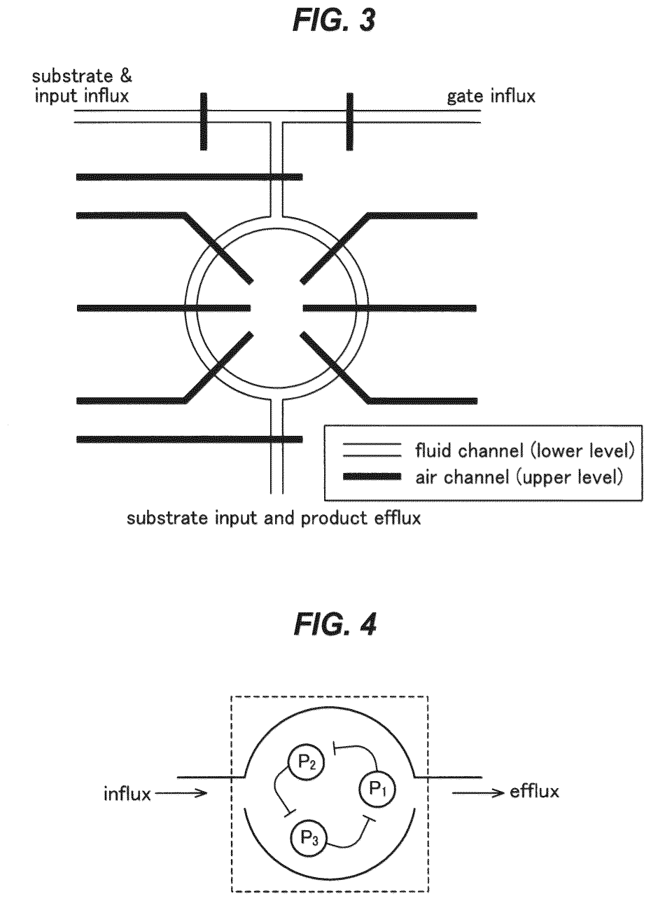

An example of implementing the above-described three-layer neural network using a DNA-based solution is the continuous stirred tank reactor (CSTR) described in Reference 2 below. FIG. 3 is a schematic diagram illustrating an implementation example of the CSTR described in Reference 2, and FIG. 4 is a schematic diagram illustrating an example of a configuration modeled after the CSTR described in Reference 3. The inside of the dotted box in FIG. 4 corresponds to the CSTR.
Reference 2: Farfel et al., “Towards practical biomolecular computers using microfluidic deoxyribozyme logic gate networks,” Lecture Notes in Computer Science, vol. 3892, pp. 38-54, 2005.
Reference 3: Goudarzi et al., “DNA Reservoir Computing: A Novel Molecular Computing Approach,” Lecture Notes in Computer Science, vol. 8141, pp. 76-89, 2013.

Finally, as a third point, how processing proceeds within the CSTR will be explained.

CSTR illustrated in FIG. 4, which includes different types of DNA molecules P1, P2, and P3, corresponds to the hidden layer of the three-layer neural network to which reservoir computing is to be applied, and the DNA molecules P1, P2, and P3 undergo mutual reactions within the CSTR.

As a result of the mutual reactions, the DNA molecules are discharged from the efflux, which is an output corresponding to the output layer. The concentration of the DNA molecules corresponds to an output signal.

Note that the computational function of the DNA molecules inside the CSTR is implemented by a DNA strand substitution reaction; alternatively, the calculation may be implemented by using another mode, for example, DNAzyme, which is DNA having the properties of an enzyme.

In the following explanation, the operation of neural networks constructed using DNA and signal processing will be explained on the basis of the contents described in References 4 and 5, and the operation of reservoir computing learning will be explained on the basis of the contents described in Reference 6.
Reference 4: Liu and “Reservoir Computing Using DNA Oscillators,” ACS Synthetic Biology 11 (2), pp. 780-787, 2022.
Reference 5: Salehi et al., “Molecular Sensing and Computing Systems”, IEEE Trans. Mol., Biol., Multi-Scale Comm. 1 (3), pp. 249-264, 2015.
Reference 6: Lukoševičius, “A Practical Guide to Applying Echo State Networks”, Lecture Notes in Computer Science, vol. 7700, pp. 659-686, 2012.

On the basis of the above, the processing by the nucleic-acid-molecule associative-memory training unit 103 illustrated in FIG. 1 will be explained.

The nucleic-acid-molecule associative-memory training unit 103 causes a nucleic acid molecule solution provided by the training-input-data specifying unit 101 corresponding to the input layer that generates the influx (input) in FIG. 4 to flow into the training CSTR, which is a CSTR for training.

The nucleic-acid-molecule associative-memory training unit 103 acquires a concentration change signal of N different DNA molecules in the training CSTR which are generated through a reaction of the DNA molecules contained in the nucleic acid molecule solution with the DNA molecules in the training CSTR. Then, the nucleic-training acid-molecule associative-memory unit 103 designates the concentration change signal as an N-dimensional vector X.

An M-dimensional vector Y_{target} provided by the training-output-data specifying unit 102, which is the output layer, satisfies the following equation (1), where the strength of the inter-unit connections from the hidden layer to the output layer is a matrix Wₒᵤₜ of the (M×N) dimension.

[Equation 1]

Y_{target}=Wₒᵤₜ·X (1)

The nucleic-acid-molecule associative-memory training unit 103 uses the N-dimensional vector X, the matrix Wₒᵤₜ, and the M-dimensional vector Y_{target} to perform ridge regression.

For example, the nucleic-acid-molecule associative-memory training unit 103 obtains the matrix Wₒᵤₜ by substituting the M-dimensional vector Y_{target} and the N-dimensional vector X in the following equation (2).

[Equation 2]

Wₒᵤₜ=(X^{T}X+λI)⁻¹X^{T}·Y_{target} (2)

In this ridge regression, Y_{target}=Y and Wₒᵤₜ=W.

Here, in order to solve Y=WX, the nucleic-acid-molecule associative-memory training unit 103 needs to solve the optimization problem of equation (3) below, in other words, to obtain W when the expression within { } in equation (3) is minimized, where A is a positive constant.

$$\begin{matrix}[ Math . 3 ] & \\ {arg min}_{w} \{ {‖ Y - WX ‖}^{2} + λ {‖ W ‖}^{2} \} & ( 3 )\end{matrix}$$

The following equation (4) is established, when the expression within { } in equation (3) is L(W).

$$\begin{matrix}[ Math . 4 ] & \\ L ( W ) = {‖ Y - WX ‖}^{2} + λ {‖ W ‖}^{2} = {( Y - WX )}^{T} ( Y - WX ) + λ {W}^{T} W = {Y}^{T} Y - 2 {X}^{T} YW + {W}^{T} {X}^{T} XW + λ {W}^{T} W & ( 4 )\end{matrix}$$

Here, when the value obtained by differentiating L (W) by W is 0, the following equation (5) is obtained, and the following equation (6) is established.

$$\begin{matrix}[ Math . 5 ] & \\ \frac{\partial }{\partial W} L ( W ) = - 2 {X}^{T} Y + 2 {X}^{T} XW + 2 λ W = 0 & ( 5 )\end{matrix}$$

$$\begin{matrix}[ Math . 6 ] & \\ W = {( {X}^{T} X + λ I )}^{- 1} {X}^{T} Y & ( 6 )\end{matrix}$$

As described above, the nucleic-acid-molecule associative-memory training unit 103 can obtain Wₒᵤₜ in the above equation (2).

Then, the nucleic-acid-molecule associative-memory training unit 103 gives the obtained Wₒᵤₜ to the nucleic-acid-molecule associative-memory processing unit 104.

The nucleic-acid-molecule associative-memory processing unit 104 is a network holding unit that holds a processing network that is a neural circuit network composed of nucleic acid molecules and reflecting the results obtained through the training by the nucleic-acid-molecule associative-memory training unit 103.

For example, the nucleic-acid-molecule associative-memory processing unit 104 hold a solution containing output calculation DNA molecules representing Wₒᵤₜ in concentration, provided from the nucleic-acid-molecule associative-memory training unit 103, as a processing CSTR, As a result, the processing CSTR is trained by supervised data.

Signal processing in the hidden layer of the three-layer neural network illustrated in FIG. 2 is implemented by the mutual reactions of DNA molecules generated inside the CSTR. Particularly, since reservoir computing learning and signal processing are implemented by DNA molecules, multiple DNA oscillators, which exhibit periodic oscillations in concentration over time, are used.

For the generation method of DNA oscillators, which exhibit periodic oscillations in concentration over time, a known method such as that described in Reference 7 below can be used.
Reference 7; K Montagne, R Plasson, Y Sakai, T Fujii, Y Rondelez, “Programming an in vitro DNA oscillator using a molecular networking strategy,” Molecular systems biology 7(1), pp. 466, 2011.

To perform learning based on reservoir computing, the nucleic-acid-molecule associative-memory training unit 103 during randomly assigns, initial the step, interlayer weights Wᵢₙ and W, among the weights Wᵢₙ between the input layer and the hidden layer, W inside the hidden layer, and Wₒᵤₜ between the hidden layer and the output layer, and calculates Wₒᵤₜ, which is the only weight that needs to be learned. Then, the nucleic-acid-molecule associative-memory training unit 103 sets the unit output of each layer as DNA concentration and embeds the weight coefficient the interlayer in connection the multiplication implemented by DNA strand substitution reactions, to compose a reservoir computing network by trained DNA.

The training-input-data specifying unit 101 can be implemented by a training-input-data specifying device including an input interface (I/F) that accepts input of training input data and a device that generates a nucleic acid molecule solution that indicates the training input data in concentration.

The training-output-data specifying unit 102 can be implemented by an input I/F that accepts input of training output data.

The nucleic-acid-molecule associative-memory training unit 103 can be implemented by a nucleic-acid-molecule associative training system including a nucleic-acid-molecule-solution holding device that holds a CSTR and causes a nucleic acid molecule solution that is training input data to flow into the CSTR, a measurement device that measures the concentration of the solution output from the CSTR after a reaction, and a computer that specifies data from the measured concentration and performs the above-described training.

The nucleic-acid-molecule associative-memory processing unit 104 can be implemented by a nucleic-acid-molecule associative device that holds a CSTR, causes a nucleic acid molecule solution that is processing input data to flow into the CSTR, and outputs the solution output from the CSTR after a reaction.

As described above, according to the first embodiment, an associative memory circuit can be constructed in the nucleic-acid-molecule associative-memory processing unit 104 by applying reservoir computing to a nucleic acid molecule network composed in a solution composed of nucleic acid molecules such as DNA and by performing associative memory training. Accordingly, data can be stored in the associative memory circuit without using base sequences on the nucleic acid molecules, and data storage can be constructed with nucleic acid molecules.

### Second Embodiment

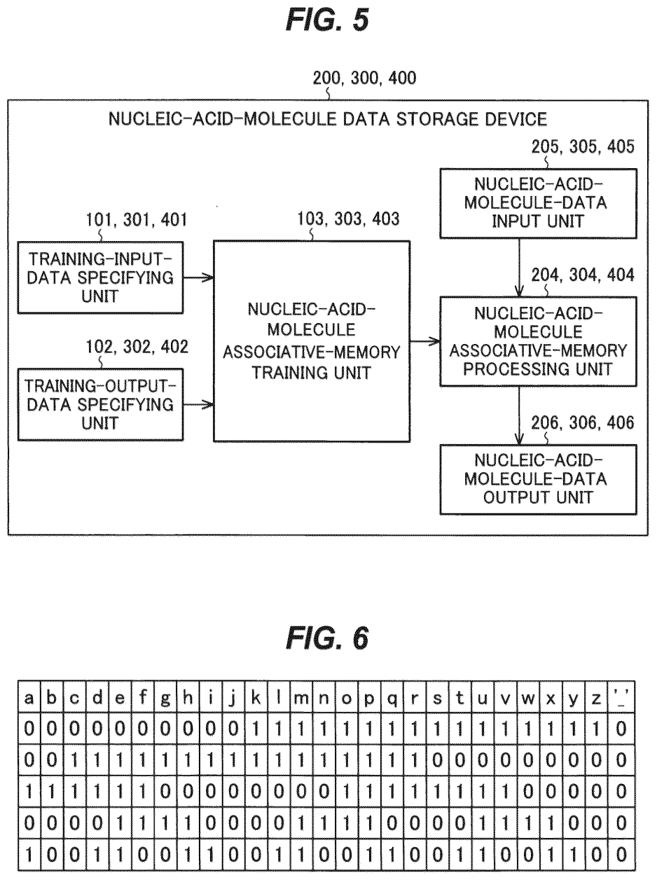

FIG. 5 is a block diagram schematically illustrating a configuration of a nucleic-acid-molecule-data storage device 200 serving as a nucleic-acid-molecule-network processing device according to the second embodiment.

The nucleic-acid-molecule-data storage device 200 includes a training-input-data specifying unit 101, a training-output-data specifying unit 102, a nucleic-acid-molecule associative-memory training unit 103, a nucleic-acid-molecule associative-memory processing unit 204, a nucleic-acid-molecule-data input unit 205, and a nucleic-acid-molecule-data output unit 206.

The training-input-data specifying unit 101, the training-output-data specifying unit 102, and the nucleic-acid-molecule associative-memory training unit 103 of the nucleic-acid-molecule-data storage device 200 according to the second embodiment are respectively the same as the training-input-data specifying unit 101, the training-output-data specifying unit 102, and the nucleic-acid-molecule associative-memory training unit 103 of the nucleic-acid-molecule-data storage device 100 according to the first embodiment.

The nucleic-acid-molecule-data input unit 205 is a processing-input-data specifying unit that specifies processing input data that is input data for performing inference.

For example, the nucleic-acid-molecule-data input unit 205 accepts processing data input, generates a nucleic acid molecule solution numerically expressing the processing input data in concentration, and gives the nucleic acid molecule solution to the nucleic-acid-molecule associative-memory processing unit 204.

The nucleic-acid-molecule associative-memory processing unit 204 hold a solution containing output calculation DNA molecules representing Wₒᵤₜ in concentration, provided from the nucleic-acid-molecule associative-memory training unit 103, as a processing CSTR.

Then, the nucleic-acid-molecule associative-memory processing unit 204 causes the nucleic acid molecule solution from the nucleic-acid-molecule-data input unit 205 to flow into the processing CSTR, allows the DNA molecules contained in the nucleic acid molecule solution to react with the DNA molecules in the processing CSTR, and outputs the solution to the nucleic-acid-molecule-data output unit 206 after the reaction.

The nucleic-acid-molecule-data output unit 206 is an output-data acquiring unit that acquires, as output data, data expressed in the concentration of the nucleic acid molecule solution obtained by inputting the nucleic acid molecular solution expressing the processing input data in concentration to the processing CSTR, which is a processing network.

For example, the nucleic-acid-molecule-data output unit 206 acquires data from the concentration of the nucleic acid molecule solution from the nucleic acid-molecule associative-memory processing unit 204 and uses the data as output data.

Specifically, the nucleic-acid-molecule-data output unit 206 acquires a concentration change signal of N different DNA molecules contained in the solution from the nucleic-acid-molecule associative-memory processing unit 204, and designates the concentration change signal as an N-dimensional vector X.

When Y_{target} is a concentration vector of M types of output DNA and Wₒᵤₜ is a concentration matrix of (M×N) output calculation DNA molecules, the nucleic-acid-molecule-data output unit 206 can calculate Y_{target} as the product of the concentration matrix Wₒᵤₜ and the concentration vector X through a reaction of the DNA molecules inside the processing CSTR and the output calculation DNA molecules. Here, the output DNA is DNA contained in the solution output from the processing CSTR, and the output calculation DNA molecules are DNA molecules contained in the nucleic acid molecule solution made to flow into the processing CSTR.

For example, pages 250 and 251 of Reference 8 below describe that equation (8) below holds when the following equation (7) is used for concentrations x, y, and z of different DNA molecules.

$$\begin{matrix}[ Math . 7 ] & \\ \begin{matrix}x + y {\to }^{{k}_{1}} x + y + z , z {\to }^{{k}_{2}} ∅ & ( {k}_{1} , {k}_{2} ; reaction rate )\end{matrix} & ( 7 )\end{matrix}$$

$$\begin{matrix}[ Math . 8 ] & \\ z = \frac{{k}_{1}}{{k}_{2}} xy & ( 8 )\end{matrix}$$

In this way, the nucleic-acid-molecule-data output unit 206 can implement multiplication by the reaction of DNA molecules, and further, according to Reference 9, can achieve multiplication of a matrix and a vector expressed by the concentrations of different DNA molecules.
Reference 8: Salehi et al., “Molecular Sensing and Computing Systems”, IEEE Trans. Mol., Biol., Multi-Scale Comm. 1(3), pp, 249-264, 2015.
Reference 9: Yang et al., “N-dimensional matrices operation based on DNA strand displacement”, ICACI 2018, pp. 490-501, 2018.

Through this operation, calculations based on reservoir computing are implemented on a recurrent neural network with the nucleic-acid-molecule-data input unit 205 as the input layer, the nucleic-acid-molecule associative-memory processing unit 204 as the hidden layer, and the nucleic-acid-molecule-data output unit 206 as the output layer.

The nucleic-acid-molecule-data input unit 205 can be implemented by a processing-data input device including an input I/F that accepts input of processing input data, and a device that generates a nucleic acid molecule solution that indicates the processing input data in concentration.

The nucleic-acid-molecule-data output unit 206 can be implemented by a nucleic-acid-molecule associative output system including a measurement device that measures the concentration of the solution output from the CSTR after a reaction, and a computer that specifies data from the measured concentration and performs the above-described training.

As described above, according to second the embodiment, when associative memory training is performed by applying reservoir computing to a nucleic acid molecule network composed in a solution composed of nucleic acid molecules such as DNA, training is performed by associating an arbitrary output pattern with the training input data, which is training data, to configure a mutual recall type associative memory circuit in the nucleic-acid-molecule associative-memory processing unit 204.

Furthermore, since the processing input data is input to the nucleic-acid-molecule associative-memory processing unit 204, and the nucleic-acid-molecule-data output unit 206 reads the concentration of the solution output by the nucleic-acid-molecule associative-memory processing unit 204, data can be read from the data storage constructed with nucleic acid molecules without reading the base sequence; thus, the processing can be sped up.

### Third Embodiment

The example described in the third embodiment describes is the same as the second embodiment except that a mutual recall type associative memory is constructed on a recurrent neural network based on reservoir computing, and multiple English words are sequentially stored. An algorithm for implementation as computer software is described.

As illustrated in FIG. 5, a nucleic-acid-molecule-data storage device 300 according to the third embodiment includes a training-input-data specifying unit 301, a training-output-data specifying unit 302, a nucleic-acid-molecule associative-memory training unit 303, a nucleic-acid-molecule associative-memory processing unit 304, a nucleic-acid-molecule-data input unit 305, and a nucleic-acid-molecule-data output unit 306.

The training-input-data specifying unit 301 specifies training input data that is input data serving as training data. The training input data according to the third embodiment will be described later.

The training-output-data specifying unit 302 specifies training output data that is output data serving as truth data. The training output data according to the third embodiment will be described later.

Supervised data is composed of the training input data specified by the training-input-data specifying unit 301 and the training output data specified by the training-output-data specifying unit 302. The supervised data according to the third embodiment will now be described.

In the third embodiment, the training output data is an example of storage data that is read by using the training input data.

As an example, in order to store 26 English words, each consisting of 9 alphabetic characters, each alphabetic character is represented by a 5-bit binary pattern.

FIG. 6 is a schematic diagram illustrating correspondence table for binarizing each alphabetic character into 5 bits.

As illustrated in FIG. 6, each character from a to z is assigned a vertical sequence of binarized signals. For example, the character a is represented by “00101,” which is a 5-bit binary number. Note that “_” in FIG. 6 is expressed as “00000” and is used for a registered character other than the 26 alphabetic characters.

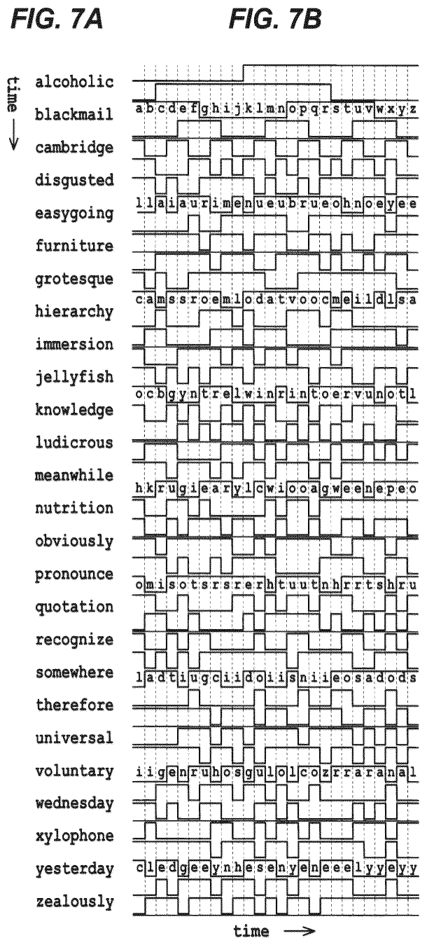

FIGS. 7A and 7B are schematic diagrams illustrating time-series binarization patterns of training input data in supervised data composed of 26 English words, each consisting of 9 characters.

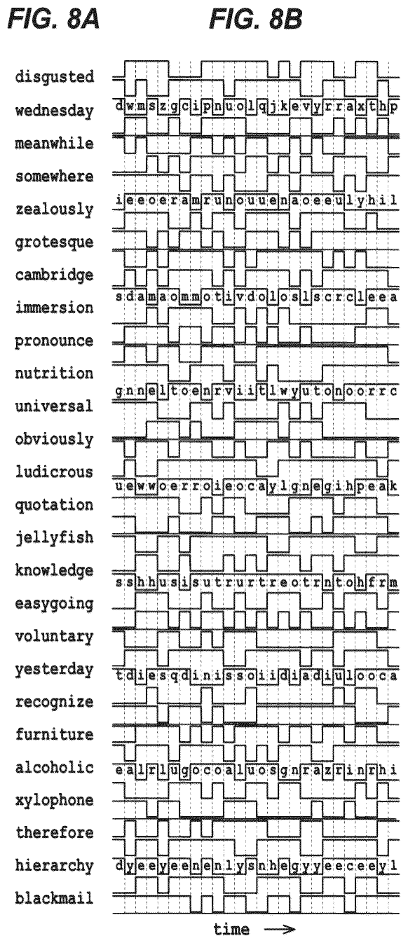

FIGS. 8A and 8B are schematic diagrams illustrating time-series binarization patterns of training output data of supervised data composed of 26 English words, each consisting of 9 characters.

Here, the data is used to train a recurrent neural network that is trained on the basis reservoir computing to learn these 26 English words for mutual recall type associative memory.

In other words, the supervised data is structured so that first training output data, corresponding to first training input data, becomes second training input data, and second training output data, corresponding to the second training input data, then becomes third training input data. Then, the first training input data is set as n-th training output data (where n is an integer of two or more) corresponding to n-th training input data, which is the last piece of supervised data, so that the first training input data to the n-th training input data sequentially inferred.

Referring back to FIG. 5, the nucleic-acid-molecule associative-memory training unit 303 inputs the signals of the 26 English words in alphabetical order and in time-series as training input data to the recurrent neural network, and trains the recurrent neural network so that the corresponding 26 English words indicated in the training output data are output in time-series.

Here, the number of units constituting the input layer, the hidden layer, and the output layer required to operate the three-layer neural network in FIG. 2 as an ESN will be described.

First, for the input layer and the output layer, it is necessary to use an alphabet represented by a 5-bit binary number per character and input words each expressed with 9 characters in parallel. For this reason, the number of units possessed by each of the input layer and the output layer is 45.

The number of units in the hidden layer poses a trade-off between the capacity for information storage and the computational demand; that is, with fewer units, the amount of information that can be stored is limited, while with more units, the computational load increases, Here, the number of units in the hidden layer is empirically set to 200.

On the basis of the above settings, the nucleic-acid-molecule associative-memory training unit 303 is able to train the three-layer neural network illustrated in FIG. 2 to learn the 26 output English words in FIGS. 8A and 8B, which is the same number as the input English words illustrated in FIGS. 7A and 7B.

In other words, the nucleic-acid-molecule associative-memory training unit 303 uses the first training output data corresponding to the first training input data as the second training input data, and performs the process of training the learning network to learn the second training output data corresponding to this second training input data iteratively up to predetermined n-th data (where n is an integer of 2 or more), so that the first training input data becomes the n-th training output data; thereby, the learning network is trained to make sequential inferences of the first training input data to the n-th training input data.

The nucleic-acid-molecule associative-memory processing unit 304 hold, as a processing CSTR, a solution containing output calculation DNA molecules representing in concentration Wₒᵤₜ, which is provided from the nucleic-acid-molecule associative-memory training unit 303. In the third embodiment, the processing CSTR for sequentially outputting multiple English words is held through the above-described training, Here, if a 9-character word is expressed in a 5-bit alphabet, one word corresponds to 45 bits. Therefore, the processing CSTR according to the third embodiment includes at least 45 different types of nucleic acid molecules of the respective bits.

In other words, the nucleic-acid-molecule associative-memory processing unit 304 inputs the nucleic acid molecule solution output from the processing network to the processing network again as a nucleic acid molecule solution of the processing input data. As a result, when a nucleic acid molecule solution with the first training input data as first processing input data is input to the processing network, the nucleic-acid-molecule associative-memory processing unit 304 infers sequentially from the first processing input data corresponding to the first training input data to n-th processing input data corresponding to n-th training input data.

The nucleic-acid-molecule-data input unit 305 accepts input of processing data, which is input data used for inference, generates a nucleic acid molecule solution numerically expressing data the processing input in concentration, and provides the nucleic acid molecule solution to the nucleic-acid-molecule associative-memory processing unit 304. The processing input data according to the third embodiment will be described later.

The nucleic-acid-molecule-data output unit 306 acquires data from the concentration of the solution from the nucleic-acid-molecule associative-memory processing unit 304 and uses the data as output data. The output data according to the third embodiment will be described later.

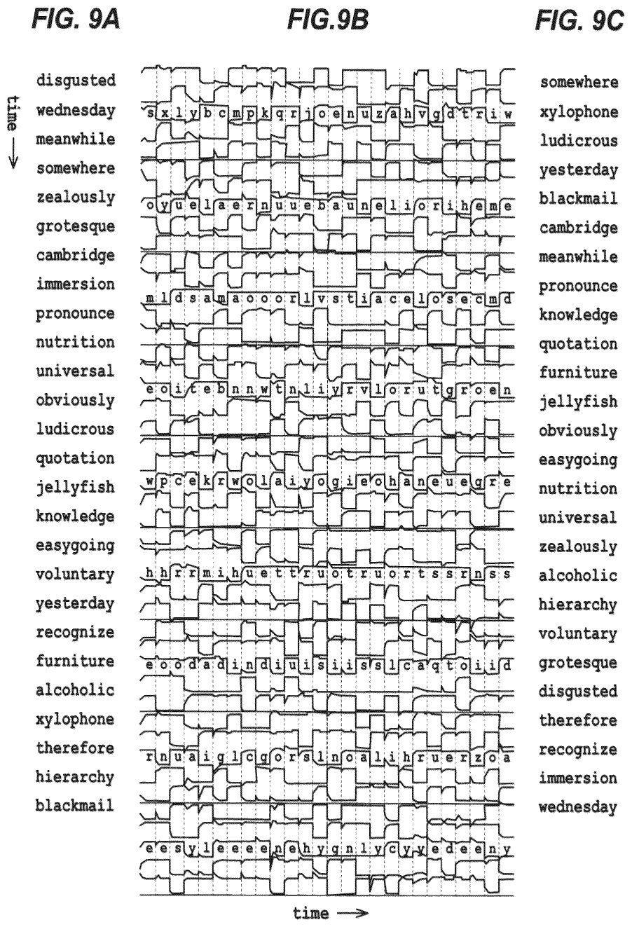

FIGS. 9A to 9C are schematic diagrams for describing the processing input data and the output data in the third embodiment.

Specifically, FIG. 9A to 9C are schematic diagrams for explaining the input to and the output from a recurrent neural network subjected to mutual recall type associative memory training based on reservoir computing.

FIG. 9A illustrates 26 English words indicated by processing input data input to the recurrent neural network; FIG. 9B illustrates signals output in response to the input of the 26 English words; and FIG. 9C illustrates output data that is examples of the result of restoring the output signals as English words.

By performing training so that when the word “alcoholic” illustrated in FIGS. 7A and 7B is input, the word “disgusted” illustrated in FIGS. 8A and 8B is output and when the word “disgusted” is input, the word “somewhere” is output, the word “somewhere” illustrated in FIGS. 9B and 9C is output when the word “disgusted” illustrated in FIG. 9A is input. By performing such processing in sequence, the output words are “disgusted,” “somewhere,” “yesterday,” “voluntary,” and then “alcoholic” are output. As described above, 26 English words can be embedded inside the three-layer neural network illustrated in FIG. 2, and each word can be sequentially retrieved.

The waveforms illustrated in FIGS. 7B, 8B, and 9B will now be described.

The signals in the network illustrated in FIG. 2 are all analog values. The circles in the input layer, the hidden layer, and the output layer illustrated in FIG. 2 are processing units defined by certain input/output functions, and the weights Wᵢₙ, W, Wₒᵤₜ, and W_{fb} are all analog values.

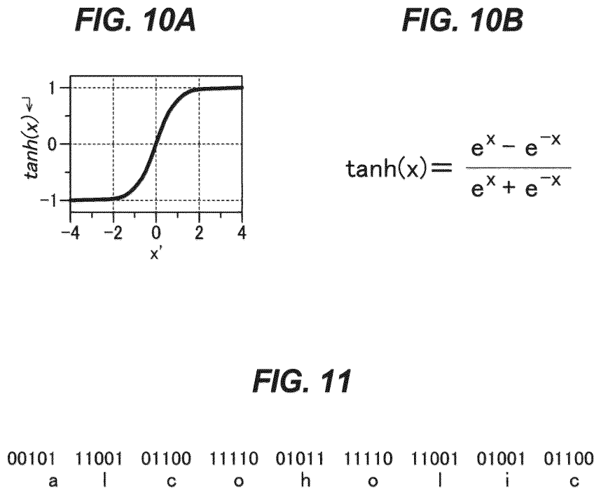

For the input/output function of each processing unit, for example, the sigmoid function tanh (x) illustrated in FIGS. 10A and 10B can be used.

The words illustrated in FIGS. 7A and 8A are supervised data to be given to the network of FIG. 2; FIG. 7A illustrates words indicated by the training input data of recall type associative memory; and FIG. 8A illustrates words indicated by training output data. In FIG. 7A, the words indicated in the training input data of the recall type associative memory are chronologically given to the network of FIG. 2 in the order indicated by the “time” arrow.

Here, when the first word “alcoholic” in FIG. 7A is input to the network, it is necessary to convert the word into binary signals. According to the conversion table of FIG. 6, the word “alcoholic” is converted into a 5×9-bit binary signal obtained by arranging 5-bit binary signals corresponding to the respective characters in “alcoholic,” as illustrated in FIG. 11.

The signal in the leftmost column of FIG. 7B is generated by encoding this binary signal as a column vector. Similarly, a 5×9-bit binary signal corresponding to the second word “blackmail” in FIG. 7A corresponds to the second column from the left in FIG. 7B. In other words, the 26 words chronologically arranged vertically in FIG. 7A are represented in FIG. 7B as single-column, 45-bit binary signals chronologically arranged horizontally. Then, the 45-bit binary signals consisting of 26 columns are input to the input layer of FIG. 2 in parallel.

The same applies to the words indicated by the training output data illustrated in FIG. 8A; for example, the first word “disgusted” is expressed by a 45-bit binary signal, which to corresponds the leftmost column illustrated in FIG. 8B. Similarly, the words arranged vertically in FIG. 8A are chronologically arranged horizontally as 45-bit column vectors in FIG. 8B.

Since the signals illustrated in both FIGS. 7B and 8B are supervised data, the signals are expressed in 0 and 1, but this only 0 and 1 as analog values, and it should be noted that the signals are not processed as binary values.

The analog waveforms illustrated in FIG. 9B will now be described.

FIGS. 9B and 9C illustrate the results of using an ESN network for which recall type associative memory training has been finished, and FIG. 9A illustrates the same 26 words as in FIG. 8A, which are here used as input to the ESN network. In other words, the 45 parallel signals illustrated in FIG. 8B are chronologically input to the trained ESN network.

The output of the ESN network at that time is the signals illustrated in FIG. 9B, and is output as analog values. In the third embodiment, for example, to express these as words, the determination of 0 or 1 is made on the basis of a threshold of 0.4, where the output of the ESN network is binarized to 1 if the value is 0.4 or more, and 0 if the value is less than 0.4. FIG. 9C illustrates the results of converting the 45-bit binary signals obtained in this way back to the alphabetic characters according to the correspondence table of FIG. 6.

As described above, according to the third embodiment, an ESN network for reading multiple words in a chain can be constructed in the nucleic-acid-molecule associative-memory processing unit 304, and by inputting processing data, multiple words can be read in a chain as output data.

### Fourth Embodiment

The example described in the fourth embodiment is the same as the second embodiment except that a mutual recall type associative memory is constructed on a recurrent neural network based on reservoir computing, and corrects an error contained an input English word when compared to a learned English word or corrects a defect in an input English word, and outputs the originally learned English word.

As illustrated in FIG. 5, a nucleic-acid-molecule-data storage device 400 according to the fourth embodiment includes a training-input-data specifying unit 401, a training-output-data specifying unit 402, a nucleic-acid-molecule associative-memory training unit 403, a nucleic-acid-molecule associative-memory processing unit 404, a nucleic-acid-molecule-data input unit 405, and a nucleic-acid-molecule-data output unit 406.

The training-input-data specifying unit 401 specifies training input data that is input data serving as training data. The training input data according to the fourth embodiment will be described later.

The training-output-data specifying unit 402 specifies training output data that is output data serving as truth data. The training output data according to the fourth embodiment will be described later.

Supervised data is composed of the training input data specified by the training-input-data specifying unit 401 and the training output data specified by the training-output-data specifying unit 402. The supervised data according to the fourth embodiment will now be described.

The fourth embodiment is an example in which the training input data and the training output data are the same data.

In the fourth embodiment, the binary patterning of an English word consisting of nine alphabetic characters is the same as that in the third embodiment, and follows the correspondence table of FIG. 6.

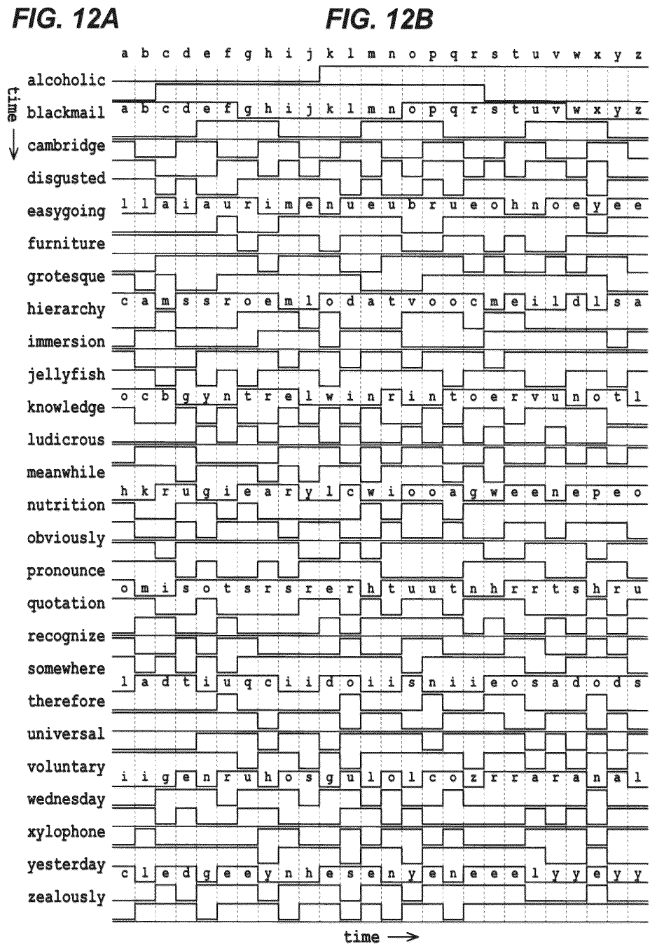

FIGS. 12A and 12B are schematic diagrams illustrating an example of training input data and training output data according to the fourth embodiment.

Specifically, FIGS. 12A and 12B illustrate 26 English words indicated by training input data and training output data consisting of 9 characters for each of input and output, which are used to train a recurrent neural network based on reservoir computing to learn English words for self-recall type associative memory, and time-series binarization patterns corresponding to the training input data and the training output data.

The difference between the mutual recall type associative memory training described in the third embodiment and the self-recall type associative memory training described in the fourth embodiment is that the former is trained so that an input pattern corresponds to an output pattern different from the input pattern, whereas the latter is trained so that the same pattern as an input pattern corresponds to the output pattern. However, there is no difference in the training algorithm using ESN. However, by using the same pattern for the output pattern as the input pattern, even if the input pattern is not identical to the learned pattern, for example, if it contains spelling mistakes or omissions, the output pattern corresponding to the original input pattern is still produced; this property results in different effects from the third embodiment.

Referring back to FIG. 5, the nucleic-acid-molecule associative-memory training unit 403 inputs the signals of the 26 English words in alphabetical order and in time-series as training input data to the recurrent neural network, and trains the recurrent neural network so that the corresponding 26 English words indicated in the training output data are output in time-series. The training operation itself is the same as that in the third embodiment.

The nucleic-acid-molecule associative-memory processing unit 404 hold a solution containing output calculation DNA molecules representing Wₒᵤₜ in concentration, provided from the nucleic-acid-molecule associative-memory training unit 303, as a processing CSTR. In the fourth embodiment, the processing CSTR for correcting defects such as errors or omissions in the input English words is held through the above training.

The nucleic-acid-molecule-data input unit 405 accepts input of processing data, which is input data used for processing, generates a nucleic acid molecule solution numerically expressing the processing input data in concentration, and provides the nucleic acid molecule solution to the nucleic-acid-molecule associative-memory processing unit 404. The processing input data according to the fourth embodiment is the same as that according to the third embodiment, but the processing input data may include defects.

The nucleic-acid-molecule-data output unit 406 acquires data from the concentration of the solution from the nucleic-acid-molecule associative-memory processing unit 304 and uses the data as output data. The output data according to the fourth embodiment will be described later.

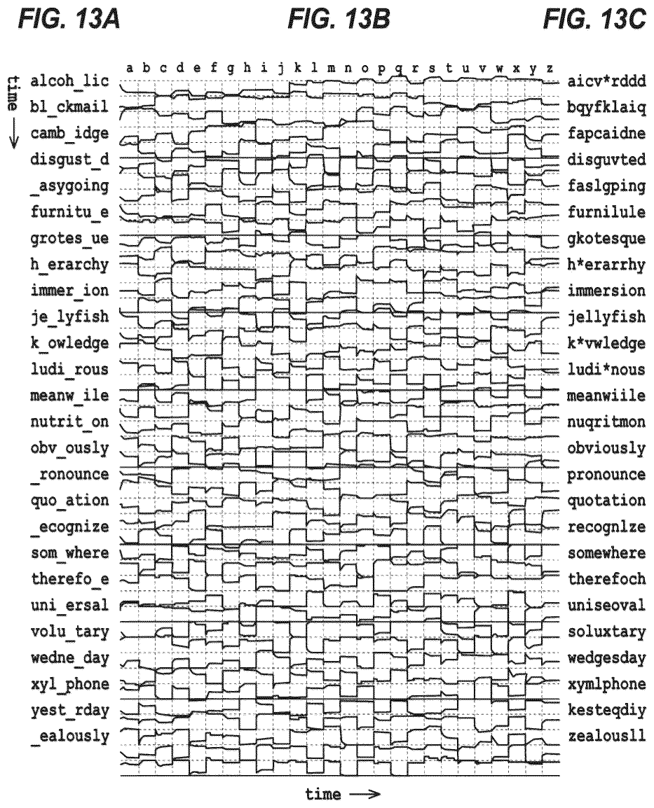

FIGS. 13A to 13C are schematic diagrams for describing the processing input data and the output data in the fourth embodiment.

Specifically, as illustrated in FIG. 13A, a recurrent neural network subjected to self-recall type associative memory learning based on reservoir computing receives input of 26 English words each missing one character, as illustrated in FIG. 13A, which correspond to the 26 English words used at the time of training, each consisting of 9 characters. In such a case, the signals as illustrated in FIG. 13B are output from the recurrent neural network as output signals, and words are restored from the output signals, as illustrated in FIG. 13C.

As a result of learning the input patterns and the output patterns in 0 and 1 at the time of learning, the signals output from the recurrent neural network after the learning become analog signals approximating 0 or 1. However, for example, when a threshold T=0.4 is set, each analog signal can be converted to a digital signal of 0 if the value is less than T and to a digital signal of 1 if the value is T or more, to approximate an English word including a missing character to the original word, but a complete word may not be restored, as illustrated in FIG. 13C.

In the fourth embodiment, for each of the 26 9-character English words used during training, if an input word with a missing character is input, multiple restorations can be performed to approach full restoration, even if full restoration is not achieved at first.

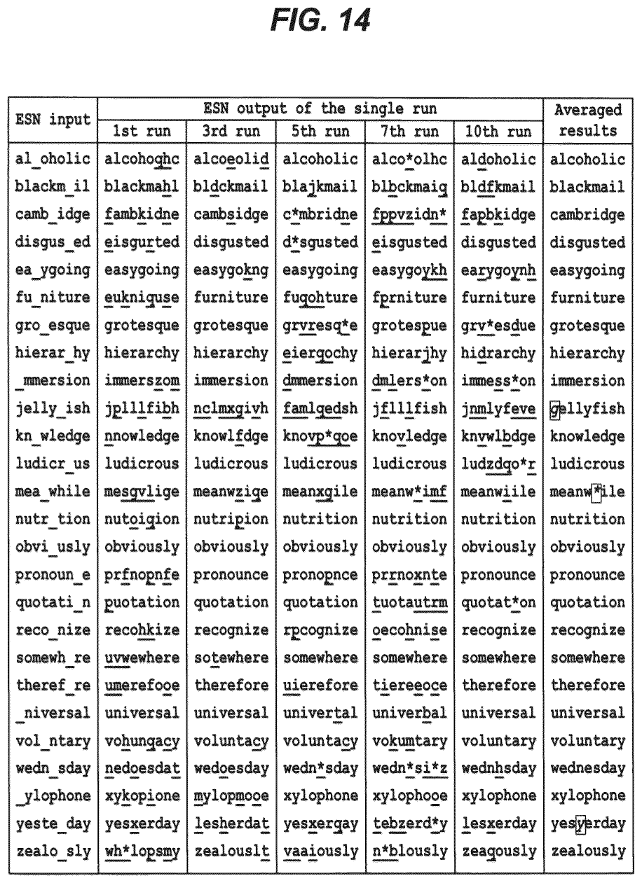

For example, FIG. 14 a schematic diagram illustrating an example of English words restored through 10 runs of restoration, as explained the above. As illustrated in FIG. 14, by averaging the signals output as a result of the 10 runs of restoration, the restored English words are closer to their full restored forms than after a single run.

As explained above, according to the fourth embodiment, even if the processing input data contains errors, in other words, even if data different from the training input data is specified as the processing input data, the properties of the self-recall type associative memory allow a correct memory pattern embedded as weights of the associative memory circuit held by the nucleic-acid-molecule associative-memory processing unit 404 to be output.

In the first to fourth embodiments, DNA is used as the nucleic molecules, but the first to fourth embodiments are not limited to such examples. For example, an associative memory network may be constructed using ribonucleic acid (RNA), which is another nucleic acid molecule, and data storage via the associative memory network by RNA may be implemented in the same way as that of DNA.

The first to fourth embodiments describe examples in which a three-layer neural network is implemented by a solution using DNA, and the DNA solution is implemented using a CSTR; however, the first to fourth embodiments are not limited to such example. For example, an associative memory network based on synthetic DNA described in Reference 10 below and a computational network related to the contents described in the first to fourth embodiments may be implemented on a silicon chip.
Reference 10: Tian et al., “Advancing high-throughput gene synthesis technology,” Molecular BioSystems vol. 5, pp. 714-722, 2009.

### DESCRIPTION OF REFERENCE CHARACTERS

100, 200, 300, 400 nucleic-acid-molecule data
storage device; 101, 301, 401 training-input-data
specifying unit; 102, 302, 402 training-output-data
specifying unit; 103, 303, 403 nucleic-acid-molecule
associative-memory training unit; 104, 204, 304, 404
nucleic-acid-molecule associative-memory processing unit;
105, 205, 305, 405 nucleic-acid-molecule-data input unit;
106, 206, 306, 406 nucleic-acid-molecule-data output unit.

1-8. (canceled)

9. A nucleic-acid-molecule-network processing device comprising:
a training-input-data specifying unit to specify training input data numerically expressed by concentration of a nucleic acid molecule solution;
a training-output-data specifying unit to specify training output data corresponding to the training input data, the training output data being numerically expressed by concentration of the nucleic acid molecule solution; and
a nucleic-acid-molecule associative-memory training unit to perform associative memory training to a neural circuit network in such a manner that the nucleic acid molecule solution numerically expressing, in concentration, the training output data specified by the training-output-data specifying unit is output when the nucleic acid molecule solution numerically expressing the training input data in concentration is input, the neural circuit network including nucleic acid molecules.

10. The nucleic-acid-molecule-network processing device according to claim 9, further comprising a nucleic-acid-molecule associative-memory processing unit to hold a result of the associative memory training performed by the nucleic-acid-molecule associative-memory training unit in a nucleic acid molecule network included in the nucleic acid molecule solution, and to compose an associative memory circuit by performing associative memory training to the nucleic acid molecule network.

11. The nucleic-acid-molecule-network processing device according to claim 10, further comprising:
a nucleic-acid-molecule-data input unit to specify processing input data numerically expressed by concentration of the nucleic acid molecule solution, and to output the processing input data to the nucleic acid molecule network in the nucleic-acid-molecule associative-memory processing unit; and
a nucleic-acid-molecule-data output unit to hold output data numerically expressed by concentration of the nucleic acid molecule solution obtained as a result of associative memory training performed by the nucleic acid molecule network that accepts input of the processing input data, and to output the output data.

12. The nucleic-acid-molecule-network processing device according to claim 9, wherein the training output data is storage data read by using the training input data.

13. The nucleic-acid-molecule-network processing device according to claim 11, wherein,
the nucleic-acid-molecule associative-memory training unit repeats, iteratively up to predetermined n-th data (where n is an integer of 2 or more), a process in which first training output data corresponding to first training input data is set as second training input data to cause the neural circuit network to perform associative memory training to learn second training output data corresponding to the second training input data, sets the first training input data as n-th training output data, thereby performing associative memory training to the neural circuit network for making sequential recall from the first training input data to the n-th training input data, and
the nucleic-acid-molecule associative-memory processing unit returns the nucleic acid molecule solution output from the nucleic acid molecule network to the nucleic acid molecule network as the nucleic acid molecule solution of the processing input data to perform associative memory training for making sequential recall of first processing input data corresponding to the first training input data to n-th processing input data corresponding to the n-th training input data when the nucleic acid molecule solution expressing the first training input data as the first processing input data is input to the nucleic acid molecule network.

14. The nucleic-acid-molecule-network processing device according to claim 9, wherein the training input data is identical to the training output data.

15. A nucleic-acid-molecule-network processing device comprising:
a nucleic-acid-molecule associative-memory processing unit to hold, in a nucleic acid molecule network, a result obtained through associative memory training performed to a neural circuit network including nucleic acid molecules in such a manner that the nucleic acid molecule solution numerically expressing training output data corresponding to the training input data in concentration is output when the nucleic acid molecule solution numerically expressing the training input data in concentration is input, and to compose an associative memory circuit by performing associative memory training to the nucleic acid molecule network;
a nucleic-acid-molecule-data input unit to specify processing input data numerically expressed by concentration of the nucleic acid molecule solution, and to output the processing input data to the nucleic acid molecule network in the nucleic-acid-molecule associative-memory processing unit; and
a nucleic-acid-molecule-data output unit to hold output data numerically expressed by concentration of the nucleic acid molecule solution obtained as a result of associative memory training performed by the nucleic acid molecule network that accepts input of the processing input data, and to output the output data.

16. A nucleic-acid-molecule-network processing method comprising:
specifying training input data numerically expressed by concentration of a nucleic acid molecule solution;
specifying training output data corresponding to the training input data, the training output data being numerically expressed by concentration of the nucleic acid molecule solution; and
performing associative memory training to a neural circuit network in such a manner that the nucleic acid molecule solution numerically expressing the training output data in concentration is output when the nucleic acid molecule solution numerically expressing the training input data in concentration is input, the neural circuit network including nucleic acid molecules.

17. A nucleic-acid-molecule-network processing method comprising:
specifying processing input data numerically expressed by concentration of the nucleic acid molecule solution;
holding, in a nucleic acid molecule network, a result obtained through associative memory training performed to a neural circuit network including nucleic acid molecules in such a manner that the nucleic acid molecule solution numerically expressing training output data corresponding to the training input data in concentration is output when the nucleic acid molecule solution numerically expressing the training input data in concentration is input, and composing an associative memory circuit by performing associative memory training to the nucleic acid molecule network; and
holding output data numerically expressed by concentration of the nucleic acid molecule solution obtained as a result of associative memory training performed by the nucleic acid molecule network that accepts input of the processing input data, and outputting the output data.
